(19) United States
(12) Patent Application Publication
(10) Pub. No.: US 2005/0107338 A1
(43) Pub. Date: May 19, 2005

(54) Nutritional supplement enhancing mitochondrial function

(76) Inventor: Michael D. Seidman, West Bloomfield, MI (US)

Correspondence Address: GIFFORD, KRASS, GROH, SPRINKLE & CITKOWSKI, P.C, PO BOX 7021, TROY, MI, 48007-7021, US

(21) Appl. No.: 10/715,148

(22) Filed: Nov. 17, 2003

(51) Int. Cl. A61K 31/685; A61K 31/385; A61K 31/198; A61K 31/05

(52) U.S. Cl. 514/78; 514/440; 514/562; 514/733

(57) ABSTRACT

A nutritional supplement for enhancing mitochondrial function, reducing age-associated DNA damage, protecting against age-associated deterioration in cognition and hearing loss and many other common deleterious changes that occur with the normal aging process is provided. The supplement includes nutritionally effective amounts of at least two components selected from the group consisting of acetyl-L-carnitine, N-acetyl cysteine, resveratrol, lecithin, and lipoic acid with the proviso that lipoic acid and acetyl-L-carnitine are not administered within the same supplement. In particular embodiments, resveratrol is present from 45 to 1000 mg in combination with one of: 600 to 3000 mg acetyl-L-carnitine, 250 to 2000 mg N-acetyl cysteine, 200 to 2000 mg lecithin, and 100 to 750 mg alpha-lipoic acid. The nutritional supplement of the present invention is preferably taken on a daily basis.

### FIELD OF THE INVENTION

This invention relates generally to methods and compositions for supplementing nutritional intake, and in particular, to methods and compositions for supplementing nutritional intake in such a way so as to enhance mitochondrial function. Most specifically, the present invention relates to a nutritional supplement containing acetyl-L-carnitine, N-acetyl cysteine, resveratrol, lecithin, and alpha-lipoic acid.

### BACKGROUND OF THE INVENTION

Aging is a progressive accumulation of metabolic and physiologic changes associated with an increasing susceptibility to disease. Several explanations for the aging process are described in the contemporary literature. Among the most prominent are the dysdifferentiation hypothesis of aging and the membrane hypothesis of aging. The dysdifferentiation hypothesis proposes that aging is the result of a continued programmed differentiation leading to either a cessation of normal gene activity or a systematic activation of genes whose effects are deleterious to cellular function. Conversely, the membrane hypothesis of aging (MHA) states that aging is related to decreasing effectiveness of cellular protective and reparative mechanisms secondary to damage from oxygen radicals. This yields biochemical and metabolic errors which progressively accumulate, resulting in cell aging and ultimately death. Therefore, the MHA suggests that reactive oxygen species (ROS) induced cell membrane structural damage is the primary mediator in cellular aging.

Reactive oxygen species (ROS), also known as free oxygen radicals (FOR), are the putative initiators in the membrane hypothesis of aging. ROS are a normal byproduct of oxidative phosphorylation, and are also formed under conditions of ischemia, hypoperfusion and because of environmental contaminants. Among the many detrimental activities of ROS, or free oxygen radicals, is direct damage to mitochondrial DNA (mtDNA). Progressive accumulation of mtDNA damage renders cells unable to conduct oxidative phosphorylation reactions effectively, thereby leading to a bioenergetically deficient cell. Over time, mitochondrial DNA damage accumulates and leads to cellular dysfunction with subsequent organ failure, aging and ultimately death. This sequence forms the basis of the MHA. Additionally, there is evidence of a reduction in the oxidant-protective enzymes superoxide dismutase and catalase associated with aging. Thus not only are there increases in the deleterious effects of ROS, but there is a reduction in the enzymes and mitochondrial metabolites necessary for protection from ROS and for effective mitochondrial function.

There is increasing support in the literature that reactive oxygen species and mitochondrial dysfunction may initiate processes that lead to Alzheimer's disease, non-specific dementias and cognitive disorders. Thus, compounds that increase mitochondrial function and scavenge or block the activities of ROS may slow or even reverse the processes of these disorders.

Aging has been shown to produce reductions in the mitochondrial membrane potentials (MMP) in rats, and studies have demonstrated reduced levels of COX and SDH with aging. Many studies have demonstrated age-associated reductions in auditory sensitivity. Specifically, reactive oxygen species (ROS) damage mtDNA leading to reduced mitochondrial function, cognition and age-related hearing loss.

Experiments have demonstrated that animals with supplementation of a mitochondrial metabolite in their drinking water for one month had improvement of their MMP, memory and general behavior (Ames B N, et al. 1983; and Gadaletta M N, et al. 1994).

Acetyl-L-carnitine is the acetyl ester of carnitine, a biological compound which plays a key role in the transport of fatty acids from the cytosol into the mitochondrial matrix of B-oxidation. Acetyl-L-carnitine modulates, through regulation of acetyl Co-A intracellular concentration, the metabolisms of sugars, lipids and amino acids, this way playing a pivotal role in cellular energy and turnover of cell membranes and proteins. Observations demonstrating a positive effect on survival were recently made on some rat populations treated with acetyl-L-carnitine. The mechanisms of such an effect are currently unknown, but they are very similar to the survival observed in rats fed a restricted caloric diet. Chronic treatment with acetyl-L-carnitine has shown enhanced stimulation of antiperoxidative systems, antagonism of the age-related effect on glucocorticoid secretion, increase in acetylcholine release and improvement in learning and memory. It has been reported that aged rat brains and hearts possessed a reduced steady state level of mitochondrial transcripts due to reduced RNA synthesis. Pretreatment of senescent rats with acetyl-L-carnitine was able to bring back the levels of mitochondrial transcripts to adult levels in a time and dose-dependent function.

Acetyl-L-carnitine is capable of restoring the integrity of the cardiac mitochondrial membrane altered by aging (specifically the cardiolipin content), thereby restoring the normal activity of cytochrome oxidase, adenine nucleotide translocase, and phosphate carrier. This allows more efficient oxidative phosphorylation, and therefore improves cardiac performance in aged animals.

N-acetyl cysteine (NAC) protects against the free radicals constantly being produced by cell mitochondria. The use of NAC seems to rescue cells through a protective effect on mitochondria, a well-known target for the action of TNF-alpha and for reactive oxygen species. NAC exerts protective effects, including extracellular inhibition of mutagenic agents from exogenous and endogenous sources, inhibition of genotoxicity of reactive oxygen species, modulation of metabolism coordinated with blocking of reactive metabolites, protection of DNA and nuclear enzymes, and prevention of the formation of carcinogen-DNA adducts. NAC has also demonstrated an effect on mutagen-induced chromosomal sensitivity assays, and on anticarcinogenicity in experimental animal models.

HIV-infected individuals and SIV-infected rhesus macaques have, on the average, decreased plasma cysteine and cystine concentrations and decreased intracellular glutathione levels. This decrease is prevented by treatment with N-acetyl cysteine (NAC). NAC caused this relative increase of CD4+ T cell numbers in spite of decreasing glutathione levels and not by increasing the glutathione level. (Kinscherf R, et al., FASEB J 8:448-451; 1994).

Resveratrol has many important biologic activities including: inhibition of lipid peroxidation; chelation of copper; free-radical scavenging; alteration of elcosanoid synthesis; inhibition of platelet aggregation; anti-inflammatory activity; vasorelaxing activity; modulation of lipid metabolism; anticancer activity; estrogenic activity; cardioprotection; and neuroprotection. (Belguendouz L, et al. Biochemical Pharmacology 53(9): 1347-55, 1997 May 9; Chanvitayapongs S, et al. Neuroreport 8(6):1499-502, 1997 Apr 14; Pace-Asciak C, et al. Clinica Chimica Acta 235(2):207-19, 1995 Mar 31; Bertelli A, et al. International Journal of Tissue Reactions 17(1):1-3, 1995; Chen C, et al. General Pharmacology 27(2):363-6, 1996 Mar.) Bertelli et al. investigated the absorption, the concentration in different organs, and the excretion of natural trans- and cis-resveratrol after red wine oral administration to rats (Bertelli A, et al. International Journal of Tissue Reactions 18(2-3):67-71, 1996). Their results show that prolonged administration of red wine in the diet could lead to an increased resveratrol concentration in different tissues even though the amount of resveratrol in these different tissues was lower than that required for pharmacological activity. This may explain its beneficial role against coronary heart disease.

Oxidative stress in the central nervous system (CNS) may cause oxidation of lipoprotein particles. The oxidized lipoproteins may damage cellular and subcellular membranes, leading to tissue injury and cell death. Draczynska-Lusiak et al. have shown that antioxidants, such as vitamins E or C, or resveratrol, protect neuronal cell damage from oxidative stress in vitro. Results indicated that oxidized lipoproteins may serve as an oxidative stressor, which may initiate the neuronal cell death leading to the manifestation of Alzheimer disease (AD) (Draczynska-Lusiak B, et al. Molecular & Chemical Neuropathology 33(2):139-48, 1998 February). Zini et al. studied the possible effects of resveratrol on the mitochondrial respiratory chain in rat brains. Resveratrol was found to decrease complex III activity in rat brain by competition with coenzyme Q (Zini R, et al. Drugs Under Experimental & Clinical Research 25(2-3):87-97, 1999). This property is especially interesting as this complex is the site where ROS are generated. By decreasing the activity of complex III, resveratrol not only opposes the production of ROS but also scavenges them (Zini R, et al. Drugs Under Experimental & Clinical Research 25(2-3):87-97, 1999). Virgili and Contestabile report that chronic administration of resveratrol to young adult rats significantly protects from the damage caused by systemic injection of the excitotoxin kainic acid in the olfactory cortex and the hippocampus (Virgili M, et al. Neuroscience Letters 281(2-3):123-6, 2000 Mar 10).

Several studies have demonstrated that antioxidants can attenuate hearing loss in various conditions, such as noise-induced hearing loss, ototoxicity, ischemia, and presbycusis. (Hatch M, et al. Hear Res 56(1-2):265-72, 1991; Komjathy D, et al. Association for Research in Otolaryngology, St. Petersburg Beach, Fla., 1998; Seidman M, et al. Otolaryngol Head Neck Surg, 109(6):1052-6, 1993; Jacono A, et al. Hear Res 117(1-2):31-8, 1998).

Lecithin is used in foods as a fat emulsifier. As a nutritional supplement, it is a natural source of many nutrients including phosphatidylcholine, phosphatidylinositol, phosphatidylserine and other phosphatides. Lecithin's most important physiological function may be its role in the biochemical processes of cell membranes, mitochondria and plasma.

Phospholipids are important compounds found in the structure of all cell membranes. Lecithin, an important source of phospholipids, is needed by every living cell in the human body. One function of cell membranes, which are composed primarily of lecithin, is to regulate the passageway of specific nutrients into or out of the cell. Without lecithin, a cell membrane would harden and become much less effective. The cell's membrane offers protection from damage by oxidation. Interestingly, the protective sheaths surrounding the brain and nerve cells contain phospholipids as found in lecithin. Additionally, muscles and nerve cells also contain this essential fatty substance. Lecithin is a rich source of the B vitamins, especially choline. Lecithin can be found in high concentrations in soybeans and egg yolks. Although lecithin is a fatty substance, it acts as an emulsifying agent, assisting in the breakdown of fats/cholesterol. It enables fats such as cholesterol and other lipids to be dispersed in water and removed from the body.

Alpha-lipoic acid is a coenzyme for the pyruvate dehydrogenase complex in the mitochondrial matrix. It is an essential cofactor for metabolism in alpha-ketoacid dehydrogenase reactions. This vitamin-like substance has been supplemented orally for health benefits and has also been used as a therapeutic agent in a variety of hepatic and neurological disorders, as well as mushroom poisoning. Consideration has also been given to the use of alpha-lipoic acid in the treatment of diabetes mellitus and atherosclerosis, in which decreased levels of alpha-lipoic acid have been found. Interestingly, a specific 10.4 kb mitochondrial DNA deletion has been found in patients with diabetes mellitus and sensorineural hearing loss. Thus, it may also be hypothesized that patients with these disorders as well as aging might benefit from a diet supplemented with lipoic acid. Dietary supplementation of alpha-lipoic acid successfully prevents myocardial damage induced by ischemia-reperfusion injury. Presently its primary therapeutic use is for the treatment of diabetic polyneuropathy.

In physiological systems, alpha-lipoic acid usually exists as lipoamide covalently attached to lysine residue of the enzyme complexes. It functions in the transfer of the two-carbon fragment from alpha-hydroxyethylthiamin pyrophosphate to acetyl-CoA, and it gets reduced in the process. The reduced form of alpha-lipoic acid is dihydrolipoic acid (DHLA) containing a disulfihydral structure. DHLA has been found to exert some antioxidant actions. It has been shown to prevent microsomal lipid peroxidation by reducing glutathione which in turn recycles vitamin E. DHLA has also been demonstrated to be a free oxygen radical scavenger to reduce peroxyl, ascorbyl and chromanoxyl radicals, and to inhibit singlet oxygen.

These compounds, due in part to their diverse effects, will synergistically act against the processes of cellular degradation through their antioxidant properties and their ability to upregulate mitochondrial function, as well as other physiologic and biochemical actions. Collectively these compounds support a situation consistent with anti-senescence. The nutritional supplement of the present invention overcomes the limitations of the prior art in that it utilizes the synergistic combination of nutritionally effective amounts of the above-referenced compounds to enhance mitochondrial function. These and other advantages of the present invention will be readily apparent from the description, discussion and experimental examples which follow.

### SUMMARY OF THE INVENTION

There is disclosed herein a nutritional supplement for enhancing mitochondrial function, reducing age-associated DNA damage, protecting against age-associated deterioration in cognition and hearing loss and many other common deleterious changes that occur with the normal aging process. The supplement includes nutritionally effective amounts of at least two components selected from the group consisting of acetyl-L-carnitine, N-acetyl cysteine, resveratrol, lecithin, and lipoic acid. In a preferred embodiment, the lipoic acid comprises alpha-lipoic acid. Lipoic acid and acetyl-L-carnitine are not administered within the same supplement.

In particular embodiments, resveratrol is present from 45 to 1000 mg in combination with one of: 600 to 3000 mg acetyl-L-carnitine, 250 to 2000 mg N-acetyl cysteine, 200 to 2000 mg lecithin, and 100 to 750 mg alpha-lipoic acid.

The nutritional supplement of the present invention is preferably taken on a daily basis; and quantities detailed herein, unless otherwise indicated, are on a daily basis.

The composition may further comprise a carrier for these components such as a liquid, tablet, capsule, patch, injectable form, intranasal route and per rectal route. The nutritional supplement of the present invention may also include ancillary ingredients such as colorings, flavorings and the like, as is known in the art.

### DETAILED DESCRIPTION OF THE INVENTION

The present invention is directed to a nutritional supplement for enhancing the mitochondrial function of cells. The supplement of the present invention enhances mitochondrial function which will physiologically and biochemically enhance energy production on a cellular level, reduce DNA damage associated with aging and improve some of the physiologic age-associated decline in energy, activity, cognition, hearing, vision, and sense of smell, etc. These changes will likely benefit people who exercise at capacity, such as athletes or individuals who exercise frequently, but will also be of benefit to individuals with relatively sedentary lifestyles and the aging population. Specifically, as we know that mitochondrial function and energy production decline with cellular degeneration associated with the normal aging process, this product will also counteract some of the biochemical processes associated with cellular degradation. Specifically, it may offer benefits to patients with cognitive disorders such as non-specific dementias, Alzheimer's disease, and individuals suffering hearing loss.

In the broadest sense, the present invention is directed to a nutritional supplement comprising the synergistic combination of a mitochondrial metabolite and a complex III blocking agent.

In one embodiment, the mitochondrial metabolite is lipoic acid, specifically alpha-lipoic acid alone or in combination with lecithin. The mitochondrial metabolite is alternatively acetyl-L-carnitine and/or glutathione biosynthesis promoter N-acetyl cysteine. A preferred cofactor is resveratrol.

In a preferred embodiment of the present invention, a nutritional supplement composition includes 25 to 2500 mg alpha-lipoic acid, 25 to 2500 mg resveratrol, 100 to 5000 mg lecithin, and 30 to 6000 mg N-acetyl cysteine. In an alternate embodiment of the present invention, acetyl-L-carnitine replaces lipoic acid.

Preferably, the nutritional supplement of the present invention is administered on a daily basis. In order to obtain the synergistic effects of the components, they may be suspended in a carrier such as a liquid, a tablet, a capsule, a transdermal patch, an injectable solution, an intranasal aerosol or a suppository, as is known in the art. It is appreciated that other routes of administration are also operative herein, these alternate routes illustratively including intracisternally, intrathecally, intravaginally, intraperitoneally, intravesically, or as a buccal or nasal spray. Ancillary ingredients such as colorings and flavorings may be added, as is well known in the art.

The present invention serves to improve mitochondrial function through the delivery of synergistic amounts of compounds active therein. While in a preferred embodiment an inventive nutritional supplement includes all of the above-described active ingredients, it is appreciated that subsets thereof are also operative to synergistically boost mitochondrial function. An improvement in mitochondrial membrane potential as a mitochondrial function measurable quantity is manifest in a variety of clinical forms illustratively including improvement in hearing, protection from noise and other ototoxic-induced hearing loss, inhibition of oxidative cellular aging processes, improvement in cognitive decline associated with neurodegenerative diseases, inhibition of age-associated DNA dilutions, and skin elasticity.

According to the present invention, subjects treated separately with resveratrol, lecithin, acetyl-L-carnitine and alpha-lipoic acid demonstrate improvement in their age-related hearing loss. Specifically, the expected deterioration in the subjects' hearing from age is statistically reduced. Additionally, the age-associated decline in mitochondrial function is improved and the increase in DNA deletions is reduced in test subjects. Furthermore, the physiologic measures of cognition are enhanced using these compounds and beneficial changes can be detected physiologically, cellularly, subcellularly and at the DNA level. Clearly these studies demonstrate the anti-senescent effects of treatment with these compounds. The effect is further enhanced when these compounds are used in combination.

The quantities of various inventive compositional components according to the present invention are shown in Table 1. Still more preferably, where less than all of the compositional components of Table 1 are present, then a mitochondrial metabolite or biosynthesis promoter thereof is present, namely lipoic acid, acetyl-L-carnitine or N-acetyl cysteine. In addition to the mitochondrial metabolite or biosynthesis promoter therefor, resveratrol and/or lecithin is present. For reasons that are as of yet unclear, the mitochondrial metabolite in the presence of resveratrol and/or lecithin appears to enhance the clinical benefits, as compared to when only a mitochondrial metabolite or a biosynthesis promoter therefor is administered.

TABLE 1
Quantity of Inventive Compositional Components.

| | General | Preferred | Optimal |
|---|---|---|---|
| Ingredient | (mg/day) | (mg/day) | (mg/day) |
| +/−Alpha lipoic acid | 25-2500 | 50-1000 | 100-750 |
| Acetyl-L-carnitine | 50-7500 | 100-5000 | 600-3000 |
| N-acetyl cysteine | 30-6000 | 100-2500 | 250-2000 |
| Resveratrol | 25-2500 | 40-1500 | 45-1000 |
| Lecithin | 100-5000 | 150-3000 | 200-2000 |

The present invention serves as a nutritional supplement to foster mitochondrial function which is manifest, for example, as retention of cognitive and auditory function through the administration by oral or parenteral routes of a large single daily dose of the synergistic compositions. It is appreciated that other routes of administration are also operative herein, these alternate routes illustratively including intracisternally, intrathecally, intravaginally, intraperitoneally, intravesically, or as a buccal or nasal spray.

Compositions suitable for parenteral injection optionally include physiologically acceptable sterile aqueous or non-aqueous solutions, dispersions, suspensions or emulsions. Examples of suitable aqueous and non-aqueous carriers, diluents, solvents and vehicles illustratively include water; ethanol; polyols, such as propylene glycol, polyethylene glycol, glycerol, and the like; combinations thereof; and injectable organic esters, such as ethyl oleate.

Therapeutic compositions optionally also include adjuvants such as preservatives, wetting agents and emulsifiers. Prevention of the action of microorganisms is assured through the addition of various antibacterial and antifungal agents, illustratively including parabens, chlorobutanol, phenol, sorbic acid, and the like. Isotonic agents are also optionally operative herein and illustratively include sugars, sodium chloride and the like.

Solid dosage forms for oral administration include capsules, tablets, pills, powders, and granules. In solid dosage forms, the therapeutic compound is admixed with at least one inert customary excipient illustratively including sodium citrate or dicalcium phosphate, or a filler, illustratively including a starch, lactose, sucrose, glucose, mannitol and silicic acid. Additionally, a binder, humectant, disintegrating agent, solution retarder, absorption accelerator, wetting agent, absorbent or lubricant is operative.

It is appreciated that in addition to an active compound, a pharmaceutically acceptable salt, ester, amide or prodrug thereof is also readily administered. Preferably, the active compound is administered without further modification.

The term “pharmaceutically acceptable salts, esters, amides, and prodrugs” as used herein refers to those carboxylate salts, amino acid addition salts, esters, amides, and prodrugs of the compounds of the present invention which are, within the scope of sound medical judgment, suitable for use in contact with the tissues of patients without undue toxicity, irritation, allergic response, and the like, commensurate with a reasonable benefit/risk ratio, and effective for their intended use, as well as the zwitterionic forms, where possible, of the compounds of the invention. The term “salts” refers to the relatively non-toxic, inorganic and organic acid addition salts of compounds of the present invention. These salts can be prepared in situ during the final isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate and laurylsulphonate salts, and the like. These may include cations based on the alkali and alkaline earth metals, such as sodium, lithium, potassium, calcium, magnesium, and the like, as well as non-toxic ammonium, quaternary ammonium and amine cations including, but not limited to ammonium, tetramethylammonium, tetraethylammonium, methylamine, dimethylamine, trimethylamine, triethylamine, ethylamine, and the like. (See, for example, Barge S M, et al., “Pharmaceutical Salts,” J Pharm Sci, 1977, 66:1-19 which is incorporated herein by reference.)

Examples of pharmaceutically acceptable, non-toxic esters of the compounds of this invention include C₁-C₆ alkyl esters wherein the alkyl group is a straight or branched chain. Acceptable esters also include C₅-C₇ cycloalkyl esters as well as arylalkyl esters such as, but not limited to benzyl. C₁-C₄ alkyl esters are preferred. Esters of the compounds of the present invention may be prepared according to conventional methods.

Examples of pharmaceutically acceptable, non-toxic amides of the compounds of this invention include amides derived from ammonia, primary C₁-C₆ alkyl amines and secondary C₁-C₆ dialkyl amines wherein the alkyl groups are straight or branched chain. In the case of secondary amines, the amine may also be in the form of a 5- or 6-membered heterocycle containing one nitrogen atom. Amides derived from ammonia, C₁-C₃ alkyl primary amines, and C₁-C₂ dialkyl secondary amines are preferred. Amides of the compounds of the invention may be prepared according to conventional methods.

In addition, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

The present invention is further illustrated with reference to the following non-limiting examples.

### EXAMPLE 1

### Mitochondrial DNA Deletion Study

A group of 154 rats are evenly divided between a control group and six study groups, resulting in 22 animals per group. The dosing for each of these groups is shown in Table 2.

TABLE 2
Study Group Dosing

| Study Group | Dosage Components | Quantity (mg/day) |
|---|---|---|
| 1 | lipoic acid | 2 |
| 2 | NAC | 15 |
| 3 | resveratrol | 10 |
| 4 | lecithin | 20 |
| 5 | 1 + 3 + 4 | 2 + 10 + 20 |
| 6 | 1 + 2 + 3 + 4 | 2 + 15 + 10 + 20 |
| 7 | control | — |

The change from baseline relative to the last study month in the levels of COX and SDH is compared among the seven study groups using one-way analysis of variance (ANOVA). The Dunnett t-test procedure is used to compare the control group to each of the six treatment groups. As a secondary analysis to evaluate for trends across time, repeated measures of ANOVA are to be used to compare the groups regarding the change in the COX and SDH levels over the three-month time intervals. If the underlying ANOVA assumptions of distributional normality or equal sample variances are violated, then data transformations are explored or a non-parametric testing alternative is utilized.

The general Chi-square test of association is used to compare the proportion of animals with mtDNA del among the seven study groups at the last study month. Then a logistic regression model is developed to evaluate the association between the last month mtDNA del status and group status after accounting for the baseline mtDNA del status.

Isolation of mitochondria from blood, brain, inner ear or other tissues is performed as previously described (Trounce, I A, et al., 1996). The sample is rinsed in cold non-ionic isolation buffer containing 210 mM mannitol, 70 mM sucrose, 1 mM EGTA, 0.5% BSA and 5 mM HEPES, pH 7.2. The resulting sample is centrifuged at 1500×g for 5 minutes at 4° C. The supernatant is decanted into a fresh tube, the pellet is discarded, and the sample undergoes repeat centrifugation. The supernatant is decanted and centrifuged at 8000×g for 15 minutes. The mitochondrial pellet is suspended in 30 ml isolation buffer and centrifuged again at 8000×g for 15 minutes. The washed mitochondrial pellet is resuspended in 0.1 ml of the isolation buffer for each gram of muscle used. This sample can then be stored at −70° C. For protein estimation, a 30 ml aliquot is removed, centrifuged at 8000×g for 15 minutes, and resuspended the pellet in isolation buffer without BSA.

Succinate dehydrogenase (SDH) does not contain any mtDNA encoded subunits; it is encoded in the nuclear DNA genome and transported across the mitochondrial membrane. The assay for SDH measures the reduction of 2,6-dichlorophenolindophenol (DCPIP) when coupled to complex 1′-catalyzed reduction of decylubiquinone (DB), monitoring the absorbance at 600 minus 750 nm (extinction coefficient 19.1 mM⁻¹cm⁻¹) (Singer T P, 1976). The reduction of DCPIP is measured at 600 minus 520 nm in a 1 ml cuvette at 30° C. Mitochondria are incubated at 30° C. for 10 minutes in a mixture of 50 mM potassium phosphate, pH 7.4, and 20 mM succinate. Antimycin A (2 μg/ml), rotenone (2 μg/ml), KCN (2 mM), and 50 μM DCPIP are added to the sample and the blank rate is recorded for 1 minute. The reaction is initiated by the addition of 50 uM DB and change in absorbance is monitored for 3 minutes.

The activity of cytochrome-c oxidase (COX) is measured by following the oxidation of reduced cytochrome c at 550 minus 540 nm (extinction coefficient 19.0 mM⁻¹ cm⁻¹) (Errede B, et al. 1996). The oxidation of ferrocytochrome c is followed at 550 minus 540 nm in a 1 ml cuvette at 300. The reduced cytochrome is added to 20 μM in 10 mM potassium phosphate, pH 7.4, and the stability of the absorbance observed for 1 minute. The reaction is initiated by adding 10 μg of mitochondrial protein. The reaction is monitored immediately and the decrease in absorbance is recorded for 30 seconds.

Blood is harvested and stored at −70° C. until the time of DNA extraction. The samples are homogenized in 10 mM Tris (pH 8.0), containing 1 mM EDTA buffer and incubated overnight at 56° C. with 15 ul Proteinase-K (10 mg/ml) in 0.5 ml digestion buffer consisting of 10 mM Tris (pH 8.0), 10 mM EDTA, 50 mM NaCl and 2% sodium-dodecyl sulfate. Standard extraction protocols for DNA are used with phenol, chloroform, and isoamyl alcohol. The proteins are removed from the sample solution with phenol:chloroform (25:24), both of which serve as separate organic solvents and hence deproteinize more efficiently. The tissue extracts are centrifuged at 10,000×g at room temperature to separate mtDNA from cellular debris, protein and genomic DNA. The supernatant is drawn off, and the residual phenol removed with equal volumes of chloroform:isoamyl alcohol (24:1). This subsequent extraction with chloroform removes the remaining traces of phenol from the preparation. 1/10 volume of 3M NaOAc and 1/100 volume of 1 M MgCl₂ are added and mtDNA is recovered by precipitation with 2.0 volumes of cold ethanol. This preparation is stored at −70° C. for 60 minutes and the precipitate will be recovered by centrifugation at 12,000×rpm for 30 minutes (4° C.). The supernatant is removed and the pellet is washed with 70% ethanol, air dried and redissolved in TE buffer at the desired concentration. Polymerase Chain Reaction (PCR) is then performed on aliquots of this DNA.

DNA concentrations are determined spectrophotometrically using optical densities of 260 and 280 nm and aliquots were used for PCR.

Oligonucleotide primers are designed in our laboratory and synthesized by Fisher Biotech (Pittsburgh, Pa.) to amplify several distinct regions of the rat, mouse and human mtDNA genome (Table 1). Of particular interest is the cytochrome b gene, the ND1 segment of the 16SrRNA region and a 4834 base pair (bp) deletion in rats, and 3726, 3867 and 4236 bp deletions in mice (Brossas J Y, et al., 1994), and 4977 bp in humans. Polymerase chain reaction (PCR) is used to amplify the base pair products that correspond to these regions. Appropriate positive and negative controls will be run and then sequenced to authenticate the PCR products.

The PCR reaction contains 100-200 ng of test sample, 200 uM of each dNTP, 50 mM KCl, 10 mM Tris-HCl (pH 8.3), 1.5 mM MgCl₂, 0.01% (wt/vol) gelatin, 1 uM of each primer and 5.0 U of Taq polymerase in a final volume of 100 ul. The thermal cycling parameters will be: Initial denaturation at 94° C. for 3 minutes, followed by 30 cycles of denaturation at 94° C. (for 30 seconds), annealing at 56° C. (for 30 seconds) and extension at 72° C. (for 1 minute). Human PCR parameters are the same with the exception of the annealing temperature which is 54° C. In mouse studies, there is no initial prolonged denaturation step. The PCR parameters for mice are 30 cycles of denaturation at 94° C. (for 20 seconds), annealing at 55° C. (for 20 seconds) and extension at 72° C. (for 90 seconds).

Study group 6 shows after one month an approximately 39% increase in COX and SDH relative to the control group (group 7). Study groups 1, 2, 3, 4 and 5 show COX and SDH averaged increases relative to the control group of 10%, 15%, 12%, 11% and 22%, respectively. The number of mitochondrial DNA deletions is lowest for study group 6 followed by that of group 5. Study groups 1-4 all showed modest decreases in mitochondrial DNA deletions relative to the control group 7.

### EXAMPLE 2

A group of human subjects are enrolled and classified into study groups in accordance with that shown in Table 2 of Example 1. Each study group is run in duplicate with the first duplicate being Alzheimer's patients while the second duplicate grouping includes like-aged individuals who do not suffer from Alzheimer's disease. Each individual prior to receipt of a dosage or placebo pill undergoes auditory testing, phlebotomy for MMP and mitochondrial DNA deletions, and cognitive testing including both Modified Mini-Mental State Examination and Digit Symbol Substitution tests.

For the seven groups of Alzheimer's patients separately from the seven groups of control patients, one-way ANOVA is used to perform the group comparisons of the change from baseline to the last study month in the levels of auditory sensitivity, cognition (total ADAS score), and MMP. The seven groups involved in this analysis are a placebo group, a group for each of the five separate drugs, and a group for the five drugs combined. The Dunnett t-test procedure is used to compare the placebo group to each of the six drug groups. As a secondary analysis to evaluate for trends across time in both settings, repeated measures of ANOVA are used to compare the groups regarding the changes overall of the monthly time points. If the underlying ANOVA assumptions of distributional normality or equal sample variances are violated, then data transformations will be explored or a non-parametric testing alternative will be utilized.

For the Alzheimer's patients separately from the control patients, the general Chi-square test of association is used to compare the proportion of humans with mtDNA del among the study groups at the last study month. Then a logistic regression model is developed to evaluate the association between the last month mtDNA del status and group status after accounting for the baseline mtDNA del status.

By using 22 patients in each of the seven groups of Alzheimer's patients and seven groups of control patients, there is a resulting power of 0.80 at an alpha level of 0.05 to detect an underlying one-way ANOVA effect size of 0.33. This corresponds to a difference between the smallest and largest group means which is about the same as the common within group standard deviation (i.e. a mean group difference of fifteen or less for the ADAS change score and a mean group difference of about 10 dB for the auditory sensitivity hearing threshold). Differences of that magnitude are meaningful for these comparisons.

This sample size results in a power of 0.80 at an alpha level of 0.05 to detect an underlying difference in deletion rates of slightly more than 0.25 in the group with the lowest deletion rate versus slightly less than 0.75 in the group with the highest deletion rate.

Human subjects are used to test the effects of mitochondrial metabolites on their hearing, cognition and mitochondrial function. Specifically, elderly human subjects undergo a detailed medical history and directed otologic examination. They have initial audiologic testing and baseline phlebotomy. They are randomized to one of the seven groups per Example 1. Their blood is drawn one month after the institution of the treatment for mitochondrial membrane potentials and at the second month of treatment the hearing is reassessed and one last phlebotomy is performed. Another protocol is used on patients with Alzheimer's disease.

The results of this study are in agreement with those obtained for rats per Example 1. Auditory sensitivity is studied using comprehensive audiometric studies including the assessment of pure tone averages, speech reception thresholds and discrimination scores. These studies are commonly performed by an audiologist in a soundproofed booth.

Mitochondrial membrane potential is measured as an average potential in isolated cells. Blood (for lymphocyte extraction) containing mitochondria is isolated from individual subjects of the seven human groups. Mitochondrial function can be measured on a cellular basis by testing their ability to generate membrane potential and hence, ATP synthesis. This is achieved by incubating cells with a fluorescent dye that is taken up exclusively by the mitochondria and accumulates into these organelles based on their membrane potential. The fluorescence level is quantified by flow cytometry and such cell sub-populations are separated by cell sorting.

In all cases, study group 6 demonstrated the most significant performance improvements in both cognitive and auditory sensitivity testing for both duplicate classes relative to the control group for each class. Additionally, the group showed the least amount of DNA damage, the highest functioning mitochondria, the best hearing and the best cognition compared to the placebo groups or the individually supplemented subjects.

All publications mentioned in the specification are indicative of the levels of those skilled in the art to which the invention pertains. These publications are herein incorporated by reference to the same extent as if each individual publication was specifically and individually indicated to be incorporated by reference.

The foregoing description is illustrative of particular embodiments of the invention, but is not meant to be a limitation upon the practice thereof. The following claims, including all equivalents thereof, are intended to define the scope of the invention.

1. A nutritional supplement comprising at least two components administered in effective daily dosages selected from the group consisting of:
25-2500 mg +/− alpha-lipoic acid,
50-7500 mg acetyl-L-carnitine,
25-2500 mg resveratrol;
100-5000 mg lecithin; and
30-6000 mg N-acetyl cysteine with the proviso that the composition lipoic acid and the acetyl-L-carnitine are not administered together.

2. The composition of claim 1 and further comprising a carrier for said components.

3. The composition of claim 1 wherein said lipoic acid comprises alpha-lipoic acid.

4. The composition of claim 2 wherein said carrier comprises a liquid.

5. The composition of claim 2 wherein said carrier is selected from the group consisting of: a capsule, a transdermal patch, an injectable solution, an intranasal aerosol and a suppository.

6. The composition of claim 2 wherein said carrier comprises a tablet.

7. The composition of claim 1 wherein said composition comprises at least one mitochondrial metabolite or promoter thereof selected from the group consisting of 25-2500 mg of lipoic acid, 50-7000 mg acetyl-L-carnitine, 30-6000 mg N-acetyl cysteine, and 40-1000 mg resveratrol.

8. The composition of claim 1 wherein said composition comprises at least one mitochondrial metabolite or promoter thereof selected from the group consisting of 25-2500 mg of lipoic acid, 50-7000 mg acetyl-L-carnitine, 30-6000 mg N-acetyl cysteine, and 150-3000 mg lecithin.

9. The composition of claim 6 wherein said lecithin is present from 100-5000 mg.

10. The composition of claim 7 wherein said resveratrol is present from 45-1000 mg.

11. A nutritional supplement comprising at least two components administered in effective daily dosages selected from the group consisting of:
600-3000 mg +/− alpha-lipoic acid,
250-2000 mg acetyl-L-carnitine,
45-1000 mg resveratrol;
200-2000 mg lecithin; and
100-750 mg N-acetyl cysteine with the proviso that the composition lipoic acid and the acetyl-L-carnitine are not administered together.

12. The composition of claim 10 and further comprising a carrier for said components.

13. The composition of claim 10 wherein said lipoic acid comprises alpha-lipoic acid.

14. The composition of claim 11 wherein said carrier comprises a liquid.

15. The composition of claim 11 wherein said carrier comprises a tablet.

16. The composition of claim 10 wherein said composition comprises at least one mitochondrial metabolite or promoter thereof selected from the group consisting of 600-3000 mg of lipoic acid, 250-2000 mg acetyl-L-carnitine, 100-750 mg N-acetyl cysteine, and 40-1000 mg resveratrol.

17. The composition of claim 10 wherein said composition comprises at least one mitochondrial metabolite or promoter thereof selected from the group consisting of 600-3000 mg of lipoic acid, 250-2000 mg acetyl-L-carnitine, 100-7500 mg N-acetyl cysteine, and 150-3000 mg lecithin.

18. The composition of claim 15 wherein said lecithin is present from 200-2000 mg.

19. The composition of claim 16 wherein said resveratrol is present from 45-1000 mg.

20. The method of promoting cognitive or auditory function, said method comprising the step of:
administering to said cells a composition comprising at least two components in synergistic effective amounts at least two components selected from the group consisting of:
25-2500 mg +/− alpha-lipoic acid,
50-7500 mg acetyl-L-carnitine,
25-2500 mg resveratrol;
100-5000 mg lecithin; and
30-6000 mg N-acetyl cysteine with the proviso that the composition lipoic acid and the acetyl-L-carnitine are not administered together.

21. The method of claim 19 wherein said step of administering occurs daily.
